(19) United States
(12) Patent Application Publication
(10) Pub. No.: US 2010/0022854 A1
(43) Pub. Date: Jan. 28, 2010

(54) Implantable Chip Medical Diagnostic Device for Bodily Fluids

(76) Inventor: Clifford Kraft, Naperville, IL (US)

Correspondence Address: CLIFFORD H. KRAFT, 320 ROBIN HILL DR., NAPERVILLE, IL, 60540, US

(21) Appl. No.: 12/504,453

(22) Filed: Jul. 16, 2009

Related U.S. Application Data
(63) Continuation of application No. 10/910,498, filed on Aug. 3, 2004.
(60) Provisional application No. 60/493,057, filed on Aug. 6, 2003.

(51) Int. Cl. A61B 5/00

(52) U.S. Cl. 600/309

(57) ABSTRACT

An implantable microchip that is attached to a source of bodily fluids such as a vein, capillary, small artery or other fluid source such as lymph fluid or urine where the fluid flows through the microchip. The microchip can contain a micro-laboratory with reagent sources and micro-canal test chambers. The microchip can contain a readout mechanism where test data is command and/or readout to an external unit. Test results can be detected with an on-chip fluorescence or light detector or an external detector.

This application is related to and claims priority from U.S. provisional application No. 60/493,057 filed Aug. 6, 2003 and hereby incorporates that application by reference.

### BACKGROUND

1. Field of the Invention

The present invention relates generally to the field of medical diagnostics and more particularly to an implantable medical diagnostic chip device.

2. Description of the Prior Art

It is known in the art to implant small electronic devices in the body. An example would be a pacemaker. It is also known to produce micro chemical analyzers on chips that have the capability to perform complex chemical or DNA analysis. In fact, it is know to provide capacity for reagents, reaction chambers and flow control totally on chips. Biochips with a quarter of a centimeter surface area that contain around a million canals with diameters of around 10 micrometers and lengths of around one half millimeter are known in the art.

### SUMMARY OF THE INVENTION

The present invention relates to a medical diagnostic device that contains at least one chip component attached to a human or animal body where at least one human bodily fluid passes through said chip component, and the chip component performs tests on the bodily fluid. Normally, the microchip device is implanted in a human or animal body. This implantation can be under the skin or deeper in the body. Many times the bodily fluid of interest is blood. An example of an application of the present invention could be in an implanted blood glucose monitor. The microchip device is normally attached to its source of fluid, such as a vein or small blood vessel by grafting or otherwise attaching the vessel so that the fluid flows through the microchip device. An alternate mode is to only sample the fluid without the fluid flowing through the microchip. The preferred mode is to have the fluid flow through the chip. The microchip device normally communicates the result of at least one test to a point outside said human body, usually some sort of collection wand or other device. The microchip device can contain a power supply which can be a battery or a power supply that is recharged from a point outside body.

### DESCRIPTION OF THE FIGURES

FIG. 1A shows a top view of an implantable microchip

FIG. 1b shows a detail view of the blood channel of the chip of Fig.

FIG. 2 shows a possible implantation and reading of a microchip.

FIG. 3A shows details of chip reagent plumbing and a possible detector.

FIG. 3B shows details of blood draw into micro-channels.

FIG. 4A shows a readout device.

FIG. 4B shows a section of the readout device of FIG. 4A.

The present invention has been described by certain figures. The scope of the present invention is not limited to what is shown in these figures.

### DETAILED DESCRIPTION OF THE INVENTION

The present invention relates to a microchip chemical analysis laboratory that can be mounted on the body or implanted in the body (possibly under the skin) that is totally self-contained and which is integrally connected to the body in such a way that a bodily fluid such as blood, lymph fluid, urine or other bodily fluid continually flows through the chip.

A common application of the present invention would be the semi-continuous or periodic monitoring of blood for glucose level for diabetes. It is very important to a diabetic to know the glucose level very frequently. Current methods require finger pricks or other methods of drawing blood. These pricks are painful, subject to infection and just plain undesirable. A micro-chip laboratory could be implanted just under the skin (or on the skin, or internally) and attached to a capillary, vein, or small artery so that blood continuously flows into and through the chip. Whenever, a glucose reading was desired, it could be performed by diverting a small amount of the blood into a test area, usually a small channel or canal, and combining this blood with a small amount of the proper reagent. A chemical or light detector could be used to read out the device. The waste blood/reagent could be stored on the chip until a possible change-out occurred. A chip with a million test canals could perform a million tests before having to be changed out. An alternative would be to discharge the waste back into the bloodstream if the reagent/blood combination was harmless. While this particular example involved blood, the same principle applies to any other bodily fluid.

Such a chip could be controlled by a resident microcontroller so that tests were performed periodically or on demand. The chip laboratory could communicate with the outside world by radio or light. The preferred method is to use a light beam that is shined onto a special liquid crystal or other surface that reflects/absorbs a specific amount of light at a particular wavelength to report the reading (in this example, glucose concentration). Alternate means of communications could be by radio, light or other wireless techniques and micro-electronics that sends and receives using known communications techniques such as pulse code modulation, phase modulation, polarization or by changing any other property of an incident or internally generated electromagnetic wave or light. Light could be internally generated by micro-LED's or by any other light generating means.

The chip laboratory including its controller and communication centers could be powered in a variety of ways. In particular, the micro-chip could contain a battery or be powered exactly like a pacemaker. However, it is also possible to replenish energy from outside the body (into an internally storage unit such as a capacitor or re-chargeable battery) with a light or radio wave where energy is taken from the incoming wave to charge the internal power source.

The micro-chip laboratory could be grafted or attached to a capillary, vein, small artery, a source of lymph fluid, or onto a source of urine so that tests could be performed periodically or on demand. Care would have to be taken (using techniques known in the art) to prevent clogging of the fluid interface or clotting of blood in the case of a capillary. It is known in the art how to graft blood vessels and other bodily fluid sources. Urine could be tapped by a tiny graft through the bladder or urethra or tube from the kidney. Lymph fluid could be tapped by grafting the device into a lymph canal.

While the micro-chip could be used on urine or lymph fluid, or on any bodily fluid (spinal fluid for example), the most likely and preferred bodily fluid is blood. There are many diseases besides diabetes where it is important to perform some sort of blood chemistry. The chip laboratory could be specially configured to perform one or more blood tests as desired.

Reagents could be stored on the chip in larger reservoirs as is known in the art. The chip would be replaced when the reagent(s) were depleted or when all the test cells had been used. The concept of many redundant test cells on the chip is important to the present invention because it is generally undesirable to discharge waste materials back into the body (although that is an alternate mode of operation of the present invention). With many different test cells designed to perform the same test (or several different tests), each test cell would be used only once.

As previously discussed, detection of the test results could be read-out with a light beam (such as might be supplied by a laser). With a plurality of similar test cells, the micro-controller (or nano-controller) would choose one for the current test. The chosen cell would be filled with fluid from that passing through one or more main chip channels, and the correct reagents would be nano-pumped into the chosen cell. After the reaction was complete, the cell could be read-out with either a chemical detector, or by putting light directly into the cell and measuring reflection or fluorescence. After the read-out was complete, the cell could be “killed” to further read-outs by adding an additional reagent that caused the response to stop. In that manner, all used cells would be “dead” in the sense that they simply contained waste matter, but would not further read-out.

There are many other read-out and detection methods known in the art including micro fiber optic sensors, chemical and electrical sensors, wave-guides with attached chemical or biological tags, and many other sensor/read-out means. It is contemplated that new read-out means will be developed in the future. All such detection and read-out means are within the scope of the present invention.

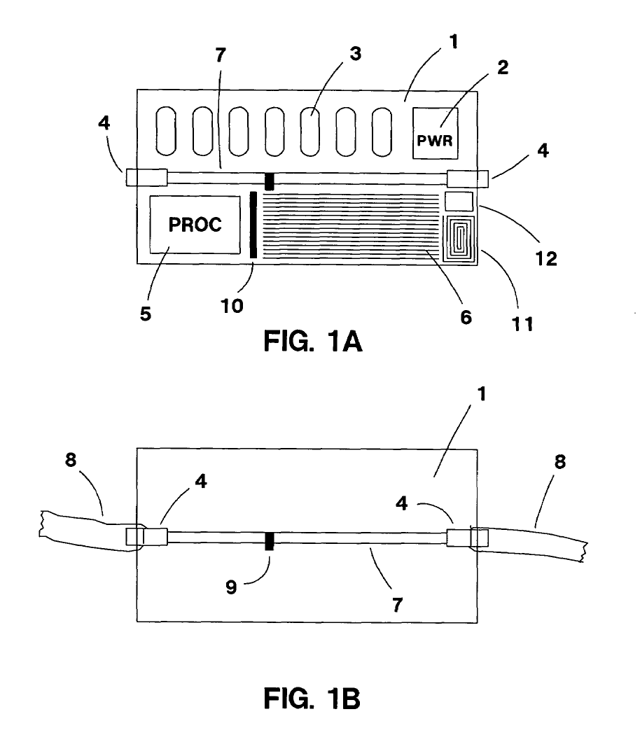

Turning to FIG. 1A, a plan view of an embodiment of the present invention is seen. The entire laboratory is mounted on an implantable chip 1 that is powered by a power source 2. The power source 2 can be a battery such as in a pacemaker or could be a device that derives power from an external light beam or electromagnetic or sound wave. Reservoirs 3 containing reagents are located on the chip and can deliver test quantities to micro-canals 6. A fluid pipe 7 (to pass the desired bodily fluid such as blood through the device passes the length of the device. Any configuration of this fluid pipe 7 is within the scope of the present invention. The fluid pipe (or pipes) has an optional end coupling 4 on each end that allows grafting or otherwise coupling to a body fluid vessel such as a small vein. The chip also contains a processor 5 that can be any special or standard microcontroller. This processor 5 controls the entire operation of the device for all testing and readout. Micro-valves 10 control reagent flow into the micro-canals 6. FIG. 1A also shows an optional radio transceiver 12 and an RF antenna 12. Generally communications with radio would use microwave frequencies and hence very small antenna structures.

FIG. 1B shows the chip of FIG. 1A with only the fluid pipe 9, the end couplings 4 and a tap-off valve 9 that takes fluid out of the pipe and routes it into a micro-channel. FIG. 1B also shows a body fluid vessel such as a blood vessel 8 attached to the fluid pipe 7 or end couplings 4. The end couplings 4 are entirely optional and can be used to make grafting or attachment easier.

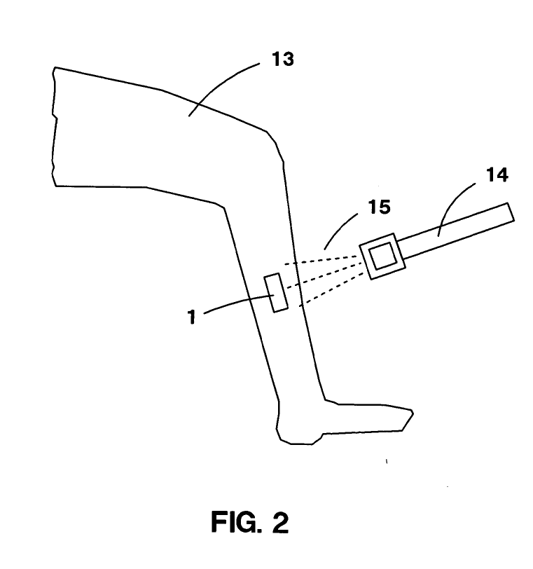

Turning to FIG. 2, the microchip 1 is seen implanted beneath the skin on a human leg 13. The microchip 1 can be mounted or implanted anywhere on a human body, wherever the desired fluid to be tested is available. FIG. 2 also shows a possible readout process where a wand device 14 is brought near the surface of the body where the microchip 1 is implanted, and a beam 15 of light or electromagnetic energy is directed into the implant. The implant can respond with the required data or be commanded to run a test. An optional readout method is to equip the microchip itself with an optical readout such as a liquid crystal or an LED lamp that could signal. Any means of readout of the implanted microchip is within the scope of the present invention.

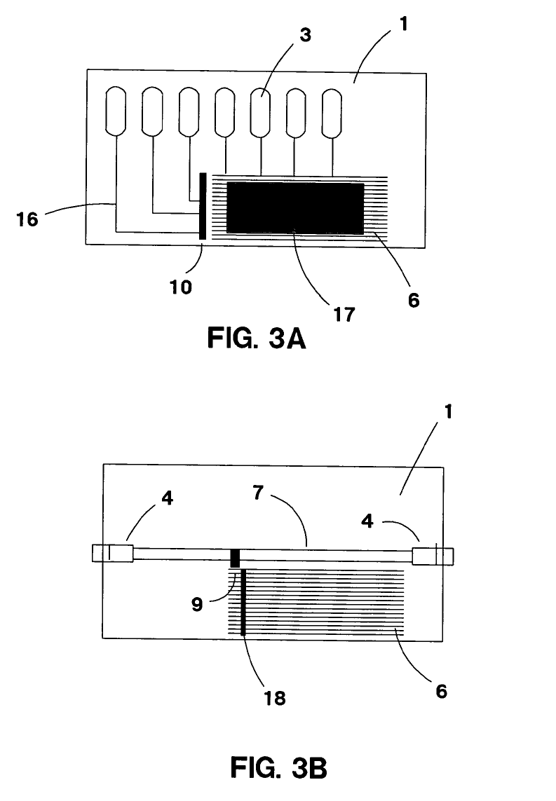

FIG. 3A shows details of reagent plumbing on the chip 1. Reagent cells 3 contain pure stock reagent for tests and are piped through micro-plumbing 16 into a valve/multiplex unit 10 that can select the proper reagent and also route it to only the currently used micro-canal. As previously stated, it is possible to have over a million micro-canals 6, each of which can be a test chamber, on a single chip. Due to complexity of reagent routing, it is possible that in some cases, reagent might be simultaneously routed to multiple canals. FIG. 3A also shows an optional optical source or detector 17 that is used for readout. On method of doing this is to use fluorescent chemicals whose light is picked up by the detector 17. An alternate method is to use a photo-excitation technique where a light source (possibly located behind the micro-canals) excites the canals, and light is picked up with an optical detector on the front.

It is not necessary to report final results, although this is the preferred method of operating the invention; rather, raw measurement data such as voltage or current could be reported with data reduction and final result computation taking place in the receiving device or in a computer.

FIG. 3B shows a detail of the fluid tap-off 9 from the fluid pipe 7 where the incoming fluid is routed to the proper micro-channel by means of a fluid router 18. Any means or method of fluid routing is within the scope of the present invention. Normally, the processor 5 (shown in FIG. 1A) can command fluid to be sampled by a particular micro-canal.

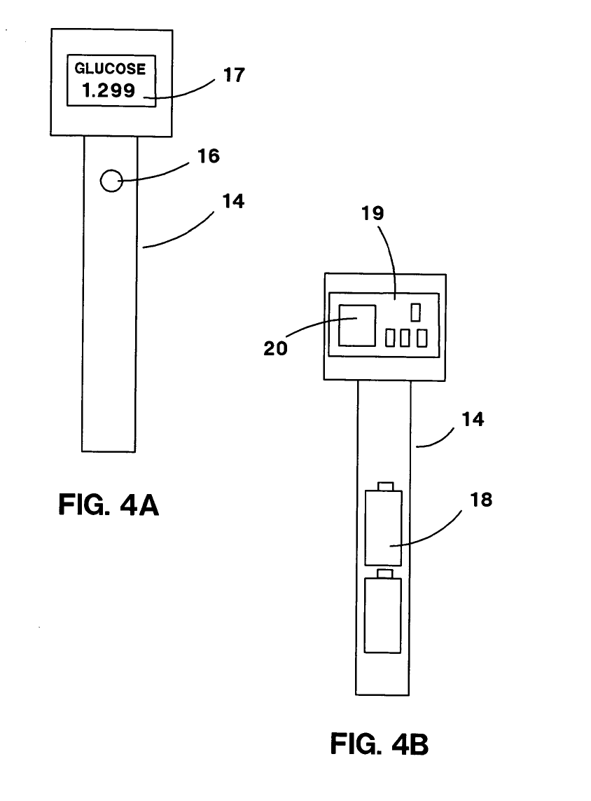

FIGS. 4A-4B show a readout wand 14 that is one of many optional ways of reading data out of the implanted microchip. In this example, the wand resembles a flashlight and contains batteries 18. A circuit board 19 that is possibly equipped with a processor causes a light or radio transceiver 20 to send out an interrogation to the implanted microchip. The answer, either raw or final data is read back, and the result is either directly displayed on an LCD or other type of display 17 or is computed and then displayed. In this example, testing and readout takes place when the wand 14 is brought near the implanted microchip, and a button 16 is pressed.

An alternate method of evaluating tests is to externally shine light onto the microchip whereby the currently used test channel is excited and then externally picking up radiated or reflected light. It is also possible to externally pick up fluorescence. This optional arrangement eliminates the need to have a detector on the

A diabetic, perhaps not feeling well, wanting to immediately access blood sugar levels could bring the wand 14 near the implanted microchip (as shown in FIG. 2), press the button 16, and have a readout within seconds. The command could cause the implanted microchip to run a real-time test (at that time), or to report data from a last periodic test. An alternate mode could be to have the microchip take periodic readings and in addition, take instant or real-time readings on command.

I claim:

1. An implantable microchip laboratory comprising:
a microchip laboratory having around a million or more micro-canal test chambers, a plurality of micro-valves, a fluid-router and a plurality of on-chip reagents on an implantable substrate, wherein said microchip laboratory performs a plurality of diagnostic tests, each test being performed in one of said micro-canal test chambers, a particular micro-canal test chamber not being reused after a test;
a fluid connection port adapted for fluid communication with at least one human bodily fluid when said microchip laboratory is implanted;
a processor also on said substrate, said processor controlling said micro-canal test chambers, micro-valves, fluid-routine and reagents in order to perform said plurality of diagnostic tests and determine results from each test so run;
a communications module non-invasively accessible when said microchip laboratory is implanted, wherein test results from each of said plurality of tests are communicated from said processor on said substrate to a remote communication unit;
a chargeable power supply also mounted on said substrate, said power supply powering at least said processor, said power supply adapted to be non-invasively recharged.

2. The implantable microchip laboratory of claim 1 wherein said micro-canals have diameters of around 10 micrometers.

3. The implantable microchip laboratory of claim 1 wherein said micro-canals of lengths of around 0.5 mm.

4. The implantable microchip laboratory of claim 1 wherein said chargeable power supply is charged using a radio wave.

5. The implantable microchip laboratory of claim 1 wherein said chargeable power supply is charged using light.

6. The implantable microchip laboratory of claim 1 wherein said micro-chip laboratory is grafted to a capillary, vein, source of lymph fluid or source of urine.

7. The implantable microchip laboratory of claim 1 wherein said communication module contains a radio transceiver.

8. The implantable microchip laboratory of claim 1 wherein fluorescent reagents are used to read out diagnostic tests in said micro-canals.

9. The implantable microchip laboratory of claim 8 further comprising a light source mounted on said substrate behind said micro-canals.

10. the implantable microchip laboratory of claim 8 wherein said microchip laboratory is read out with a hand-held wand.

11. An implantable microchip laboratory comprising:
a microchip laboratory having around a million or more micro-canal test chambers, a plurality of micro-valves, a fluid-router and a plurality of on-chip reagents on an implantable substrate, wherein said microchip laboratory performs a plurality of diagnostic tests, each test being performed in one of said micro-canal test chambers, a particular micro-canal test chamber not being reused after a test;
a fluid connection port adapted for fluid communication with at least one human bodily fluid when said microchip laboratory is implanted;
a processor also on said substrate, said processor controlling said micro-canal test chambers, micro-valves, fluid-routine and reagents in order to perform said plurality of diagnostic tests and determine results from each test so run;
a communications unit on said substrate adapted to non-invasively communicate test results from said micro-canals to a remote location;
a test result detection system also on said substrate in electrical communication with said processor;
a rechargeable charge storage device also on said substrate supplying power to said processor, charge storage device being non-invasively rechargeable.

12. The implantable microchip laboratory of claim 11 wherein said micro-chip laboratory is grafted to a capillary, vein, source of lymph fluid or source of urine.

13. The implantable microchip laboratory of claim 11 wherein said communication means contains a radio transceiver.

14. The implantable microchip laboratory of claim 11 wherein said test detection system uses fluorescent reagents to read out diagnostic tests in said micro-canals.

15. The implantable microchip laboratory of claim 11 further comprising a light source on said substrate behind said micro-canals.

16. The implantable microchip laboratory of claim 11 wherein said microchip laboratory is read-out with a hand-held wand.
